(19) United States
(12) Patent Application Publication
(10) Pub. No.: US 2022/0277844 A1
(43) Pub. Date: Sep. 1, 2022

(54) ORDER MANAGEMENT METHOD AND PROGRAM, ORDER MANAGEMENT SYSTEM, AND DATABASE FOR MEDICAL PRACTICES

(71) Applicant: FUJIFILM Corporation, Tokyo (JP)

(72) Inventor: Hironori MATSUMASA, Tokyo (JP)

(73) Assignee: FUJIFILM Corporation, Tokyo (JP)

(21) Appl. No.: 17/745,840

(22) Filed: May 16, 2022

Related U.S. Application Data
(63) Continuation of application No. PCT/JP2020/041870, filed on Nov. 10, 2020.

(30) Foreign Application Priority Data
Nov. 19, 2019 (JP) 2019-208743

(51) Int. Cl. G16H 40/20; G06Q 10/08; G16H 10/00

(52) U.S. Cl. CPC G16H 40/20; G06Q 10/087; G16H 10/00

(57) ABSTRACT

Provided are an order management method and program, an order management system, and a database for medical practices for appropriately managing orders for medical practices. An order management method for medical practices includes: a medical practice management step of managing a calculation process regarding a disease, a target part of the calculation process, a first medical practice necessary for executing the calculation process, and a second medical practice necessary for at least one of a diagnosis or treatment of the disease by a doctor in association with each other; a target part acquisition step of acquiring the target part to be examined in a patient; and an order management step of issuing an order for the first medical practice and the second medical practice at the acquired target part.

### CROSS-REFERENCE TO RELATED APPLICATIONS

The present application is a Continuation of PCT International Application No. PCT/JP2020/041870 filed on Nov. 10, 2020 claiming priority under 35 U.S. § 119(a) to Japanese Patent Application No. 2019-208743 filed on Nov. 19, 2019. Each of the above applications is hereby expressly incorporated by reference, in its entirety, into the present application.

### BACKGROUND OF THE INVENTION

### 1. Field of the Invention

The present invention relates to an order management method and program, an order management system, and a database for medical practices, and particularly relates to a technique for managing orders for medical practices.

### 2. Description of the Related Art

In the medical field, the efficiency of image interpretation and diagnostic support are being realized by using artificial intelligence (AI) using a neural network trained by deep learning.

In addition, techniques for managing medical practice orders are known. JP5252263B describes a knowledge database capable of comprehensively converting symptoms, examinations, and report contents into a database, determining whether the order is appropriate based on an existing protocol in the hospital in a case where an attending physician orders image capturing for a specific symptom, and pointing out missing of a specific examination in a case where the specific examination is missing. According to this knowledge database, for example, in the case of chest pain, in a case where a medical facility has a protocol for ordering both X-ray computed tomography (CT) and magnetic resonance (MR) examinations, it becomes possible to point out that the examination by MR is missing in a case where a healthcare professional such as a doctor orders only an X-ray CT examination based on the symptom.

### SUMMARY OF THE INVENTION

The amount of medical data to be diagnosed and the amount of medical data that needs to be managed are steadily increasing, which hinders the reduction of the burden on doctors. In order to reduce the burden on doctors, it is necessary to manage the medical data that occurs, and for that purpose, it is necessary to support the implementation management of medical practices.

The present invention has been made in view of such circumstances, and an object thereof is to provide an order management method and program, an order management system, and a database for medical practices for appropriately managing medical practice orders.

One aspect of an order management method for medical practices for achieving the above object is an order management method for medical practices comprising: a medical practice management step of managing a calculation process regarding a disease, a target part of the calculation process, a first medical practice necessary for executing the calculation process, and a second medical practice necessary for at least one of a diagnosis or treatment of the disease by a doctor in association with each other; a target part acquisition step of acquiring the target part to be examined in a patient; and an order management step of issuing an order for the first medical practice and the second medical practice at the acquired target part. According to the present aspect, the order for medical practices can be appropriately managed.

It is preferable that the order management method for medical practices further comprises a calculation process result acquisition step of acquiring a calculation process result of the calculation process regarding the disease executed based on a result of the first medical practice, and in the order management step, a necessity of the second medical practice is determined based on the calculation process result. Accordingly, the order for the second medical practice can be managed.

It is preferable that in the order management step, the order for the second medical practice is canceled in a case where the calculation process result is negative for the disease. Accordingly, it is possible to prevent unnecessary medical practices from performing and reduce the amount of data.

It is preferable that in the medical practice management step, the calculation process, the target part, the first medical practice, and the second medical practice are managed in association with each other for each of a plurality of different diseases. Accordingly, it possible to appropriately manage the orders for medical practices corresponding to a plurality of different diseases.

It is preferable that in the order management step, in a case where there are a plurality of the calculation processes corresponding to the acquired target part, orders for a plurality of first medical practices necessary for executing the plurality of calculation processes are issued in order of priority. Accordingly, it is possible to give priority to the calculation process having a high priority.

It is preferable that in the order management step, the order for the second medical practice is issued after the orders for the plurality of first medical practices. Accordingly, a plurality of calculation processes can be prioritized.

It is preferable that the calculation process is a process of performing calculation using artificial intelligence. In addition, it is preferable that the process of performing calculation using the artificial intelligence is a process of outputting an inference result for input data by a learning model trained by machine learning. Accordingly, it is possible to acquire a calculation process result obtained by appropriately calculating the result of the first medical practice.

The present aspect also includes a program for causing a computer to execute the order management method for medical practices. The program for causing a computer to execute the order management method for medical practices may be provided by being stored on a non-transitory computer-readable recording medium. According to the present aspect, the order for medical practices can be appropriately managed.

One aspect of an order management system for medical practices for achieving the above object is an order management system for medical practices comprising: a medical practice management unit that manages a calculation process regarding a disease, a target part of the calculation process, a first medical practice necessary for executing the calculation process, and a second medical practice necessary for at least one of a diagnosis or treatment of the disease by a doctor in association with each other; a target part acquisition unit that acquires the target part to be examined in a patient; and an order management unit that issues an order for the first medical practice and the second medical practice at the acquired target part. According to the present aspect, the order for medical practices can be appropriately managed.

One aspect of an order management system for medical practices for achieving the above object is an order management system for medical practices comprising: a memory that stores instructions for causing a processor to execute the instructions; and the processor that executes the instructions stored in the memory, in which the processor is configured to manage a calculation process regarding a disease, a target part of the calculation process, a first medical practice necessary for executing the calculation process, and a second medical practice necessary for at least one of a diagnosis or treatment of the disease by a doctor in association with each other, acquire the target part to be examined in a patient, and issue an order for the first medical practice and the second medical practice at the acquired target part. According to the present aspect, the order for medical practices can be appropriately managed.

One aspect of a database for medical practices for achieving the above object is a database for medical practices, comprising a calculation process regarding a disease, a target part of the calculation process, a first medical practice necessary for executing the calculation process, and a second medical practice necessary for at least one of a diagnosis or treatment of the disease by a doctor which are stored in association with each other. According to the present aspect, the order for medical practices can be appropriately managed.

According to the aspects of the present invention, the order for medical practices can be appropriately managed.

### BRIEF DESCRIPTION OF THE DRAWINGS

FIG. 1 is a block diagram showing a configuration of an in-hospital system 10.

FIG. 2 is a diagram showing an example of a table.

FIG. 3 is a block diagram showing a functional configuration of an order management system for medical practices.

FIG. 4 is a block diagram showing a main functional configuration of a calculation processing unit.

FIG. 5 is a flowchart showing an order management method for medical practices.

FIG. 6 is a table showing orders managed by an order management unit.

FIG. 7 is a table showing orders managed by the order management unit.

### DESCRIPTION OF THE PREFERRED EMBODIMENTS

Hereinafter, a preferred embodiment of the present invention will be described in detail with reference to the accompanying drawings.

### In-Hospital System

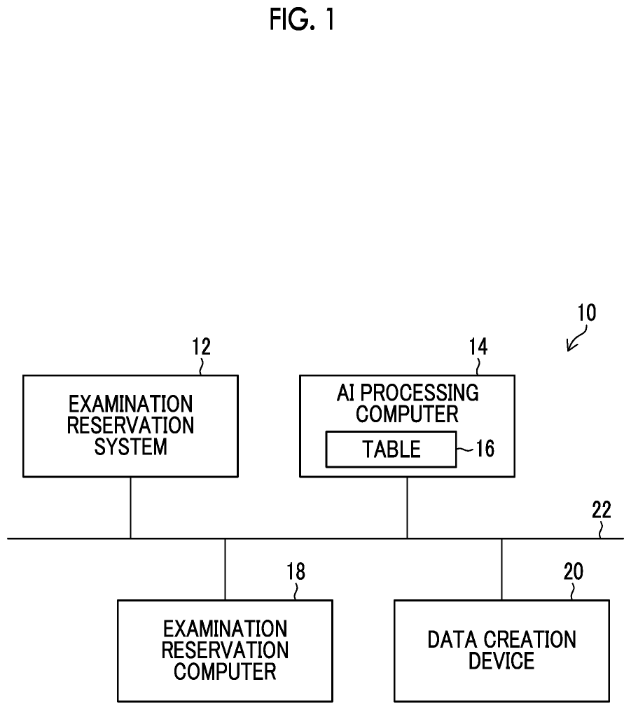

FIG. 1 is a block diagram showing a configuration of an in-hospital system 10. The in-hospital system 10 comprises an examination reservation system 12, an artificial intelligence (AI) processing computer 14, an examination reservation computer 18, a data creation device 20, and a network 22.

The examination reservation system 12 is configured with a computer and software (not shown) that manages reservations (orders) of the data creation device 20.

The AI processing computer 14 and the examination reservation computer 18 are computers used in the hospital, respectively. The AI processing computer 14 and the examination reservation computer 18 each include a hardware configuration (not shown) such as a central processing unit (CPU), a memory, a storage, an input/output interface, a communication interface, an input device, a display device, and a data bus. Further, the AI processing computer 14 and the examination reservation computer 18 each have a keyboard, a mouse, or the like (not shown) as an input device, and a display or the like (not shown) as a display device. A well-known operating system or the like is installed in each of the AI processing computer 14 and the examination reservation computer 18, and a viewer application for displaying a medical image is executed.

The AI processing computer 14 executes a plurality of calculation processes (AI processing) corresponding to a plurality of different diseases, respectively. A calculation process regarding diseases is an example of artificial intelligence, and is a learning model trained by machine learning to output inference results for input data. Input data includes medical data. The medical data is data related to a subject, and is data including at least one of medical images, pathological images, diagnostic information, or finding information. The diagnostic information includes information such as genome analysis results, electrocardiogram waveform data, and vital data. The finding information includes text data indicating the type and progress status of the disease and the like. The medical data may include personal information such as the gender and age of the subject, and clinical information such as medical history.

The AI processing computer 14 comprises a table 16. In the table 16 (an example of a database for medical practice), a target part of the calculation process, a first medical practice necessary for executing the calculation process, and a second medical practice necessary for at least one of the diagnosis or treatment of the disease by a doctor are associated and stored for each of the calculation processes that can be executed by the AI processing computer 14.

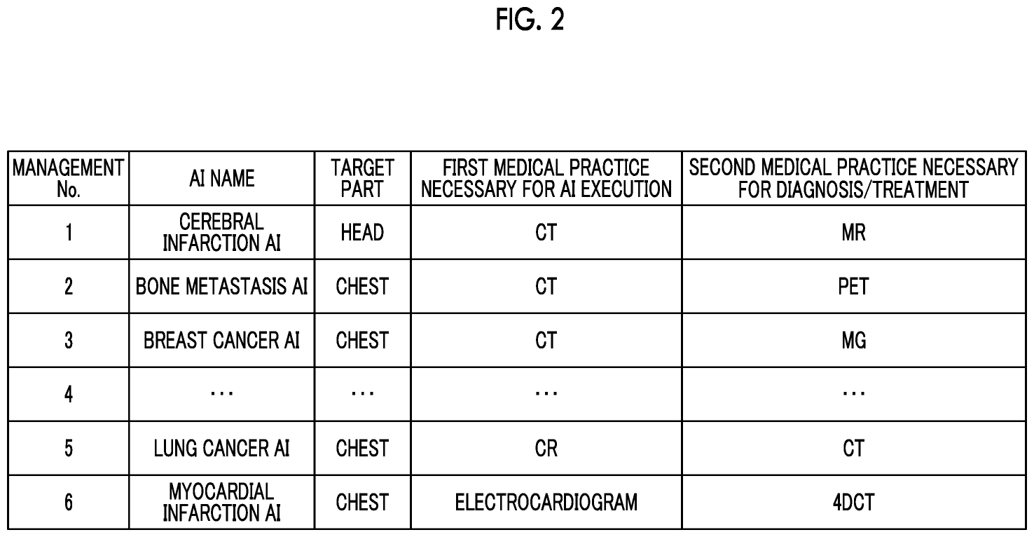

FIG. 2 is a diagram showing an example of the table 16. Here, the associated data of management numbers 1 to 6 are shown. For example, in management number 1, it is stored that the AI name is cerebral infarction AI that outputs the inference result of cerebral infarction, the target part of cerebral infarction AI is the head, the first medical practice is the capturing of a computed tomography (CT) image, and the second medical practice is the capturing of a magnetic resonance (MR) image. Further, in management number 2, it is stored that the AI name is bone metastasis AI that outputs the inference result of bone metastasis, the target part of bone metastasis AI is the chest, the first medical practice is the capturing of a computed tomography (CT) image, and the second medical practice is the capturing of a positron emission tomography (PET) image. In the present embodiment, the table 16 is used to appropriately manage the order for medical practices.

Returning to the description of FIG. 1, the examination reservation computer 18 is a computer for a doctor to place an order for an examination. The order input in the examination reservation computer 18 is transmitted to the examination reservation system 12. The examination reservation system 12 manages the order of the data creation device 20 based on the order received from the examination reservation computer 18.

The data creation device 20 is a medical information system. The data creation device 20 acquires medical information from various modalities and creates data to be used for calculation processes executed by the AI processing computer 14. The modality includes an apparatus that generates a medical image representing a target part of the subject by imaging the target part, adds accessory information specified by the DICOM standard to the medical image, and outputs the medical image. As specific examples, a computed tomography apparatus (CT apparatus), a 4 dimensions computed tomography apparatus (4DCT apparatus), a magnetic resonance imaging apparatus (MRI apparatus), a positron emission tomography apparatus (PET apparatus), an ultrasound diagnostic apparatus, a computed radiography apparatus (CR apparatus) using a flat panel detector (FPD), a mammography apparatus (MG apparatus), and the like can be mentioned.

The data creation device 20 may treat a pathological image captured by a camera of a tissue collected from a subject as a medical image. In addition, the data creation device 20 may acquire data from an electrocardiogram measuring device, an endoscope, and a genome analysis system. The electrocardiogram measuring device is a device that detects changes in the electrical activity of the heart via electrodes on the surface of the living body and records the results in the form of a graph. An endoscope is an optical system device for observing a luminal region such as the esophagus and intestine of a subject. The endoscope includes an optical system device for observing the inside of the incision part. The genome analysis system is a system that analyzes genetic information of a subject. The genome analysis system performs genome analysis on a cell sample of a subject.

The network 22 is realized by, for example, a local area network (LAN). The examination reservation system 12, the AI processing computer 14, the examination reservation computer 18, and the data creation device 20 are connected via the network 22. The function of the AI processing computer 14 may be disposed on the cloud.

### Order Management System for Medical Practices

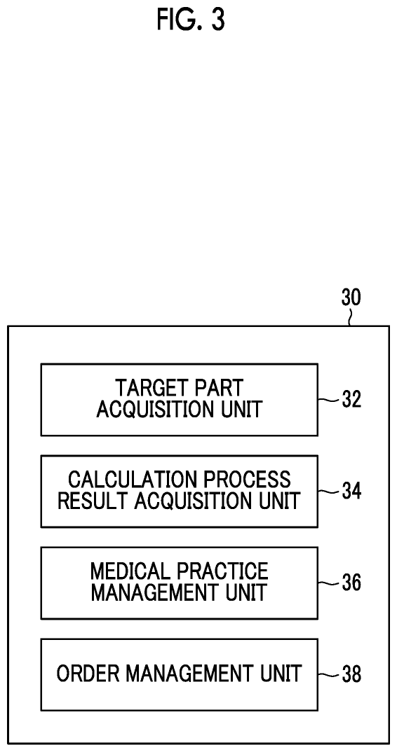

FIG. 3 is a block diagram showing a functional configuration of an order management system 30 for medical practices used in the in-hospital system 10. The processing of the order management system 30 for medical practices is performed by, for example, the examination reservation computer 18. The order management system 30 for medical practices comprises a target part acquisition unit 32, a calculation process result acquisition unit 34, a medical practice management unit 36, and an order management unit 38.

The target part acquisition unit 32 acquires a target part to be examined in a patient (subject). The calculation process result acquisition unit 34 acquires the calculation process result of the calculation process regarding the disease executed based on the result of the first medical practice.

The medical practice management unit 36 manages the calculation process regarding the disease, a target part of the calculation process, the first medical practice necessary for executing the calculation process, and the second medical practice necessary for at least one of the diagnosis or treatment of the disease by a doctor in association with each other. The order management unit 38 issues an order for the first medical practice and the second medical practice at the target part, and requests the issued order to the data creation device 20.

### Calculation Processing Unit

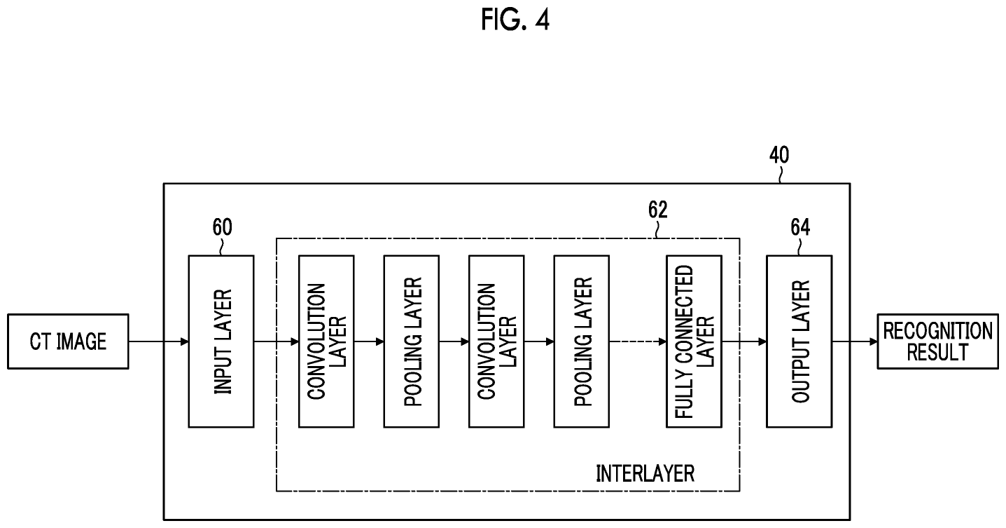

FIG. 4 is a block diagram showing a main functional configuration of the calculation processing unit 40 included in the AI processing computer 14. Here, a case where the calculation processing unit 40 is cerebral infarction AI that recognizes cerebral infarction will be described as an example. The cerebral infarction AI is a learning model that performs a recognition process of cerebral infarction using CT images, and is a learning model that learns using CT images.

The calculation processing unit 40 has a plurality of layer structures and holds a plurality of weight parameters. The calculation processing unit 40 can change from an unlearned model to a trained model by updating the weight parameter from the initial value to the optimum value.

The calculation processing unit 40 has a configuration of a convolutional neural network (CNN), and comprises an input layer 60, an interlayer 62, and an output layer 64. The input layer 60, the interlayer 62, and the output layer 64 each have a structure in which a plurality of “nodes” are connected by “edges”.

A CR image and a CT image are input to the input layer 60 as input data.

The interlayer 62 is a layer for extracting features from an image input from the input layer. The interlayer 62 has a plurality of sets including a convolution layer and a pooling layer as one set, and a fully connected layer. The convolution layer performs a convolution operation using a filter on a node nearby the preceding layer, and acquires a feature map. The pooling layer reduces the feature map output from the convolution layer to acquire a new feature map. The fully connected layer connects all the nodes of the immediately preceding layer (here, the pooling layer). The convolution layer plays a role of feature extraction such as edge extraction from an image, and the pooling layer plays a role of imparting robustness so that the extracted features are not affected by translation or the like. The interlayer 62 is not limited to the case where the convolution layer and the pooling layer are set as one set, and the convolution layer may be continuous or may include a normalization layer.

The output layer 64 is a layer that outputs a recognition result for detecting lung cancer based on the features extracted by the interlayer 62. Further, the recognition result that classifies whether the detected lung cancer is benign or malignant may be output.

In the case of classifying cerebral infarction, the trained calculation processing unit 40 classifies the recognition results into three categories, for example, “malignant”, “benign”, and “other”, and outputs the recognition result as three scores corresponding to “malignant”, “benign”, and “other”. The total of the three scores is 100%.

Any initial values are set for the coefficient of the filter applied to each convolution layer of the calculation processing unit 40 before learning, the offset value, and the weight of the connection with the next layer in the fully connected layer.

The calculation processing unit 40 adjusts weight parameters of the cerebral infarction AI by the error back propagation method based on an error between the recognition result output from the output layer 64 and correct answer data. This parameter adjustment process is repeated, and learning is repeated until the difference between the output of the cerebral infarction AI and the correct answer data becomes small.

The calculation processing unit 40 may not use the correct answer data depending on the recognition process to be realized. In addition, the calculation processing unit 40 may extract features by an algorithm designed in advance such as edge extraction, and use the information to learn with a support vector machine or the like.

The cerebral infarction AI may be trained using a CT image and an image other than the CT image, and recognition process may be performed using the CT image. That is, the input data used for the recognition process may be a subset of the input data used for the learning process.

### Order Management Method for Medical Practices

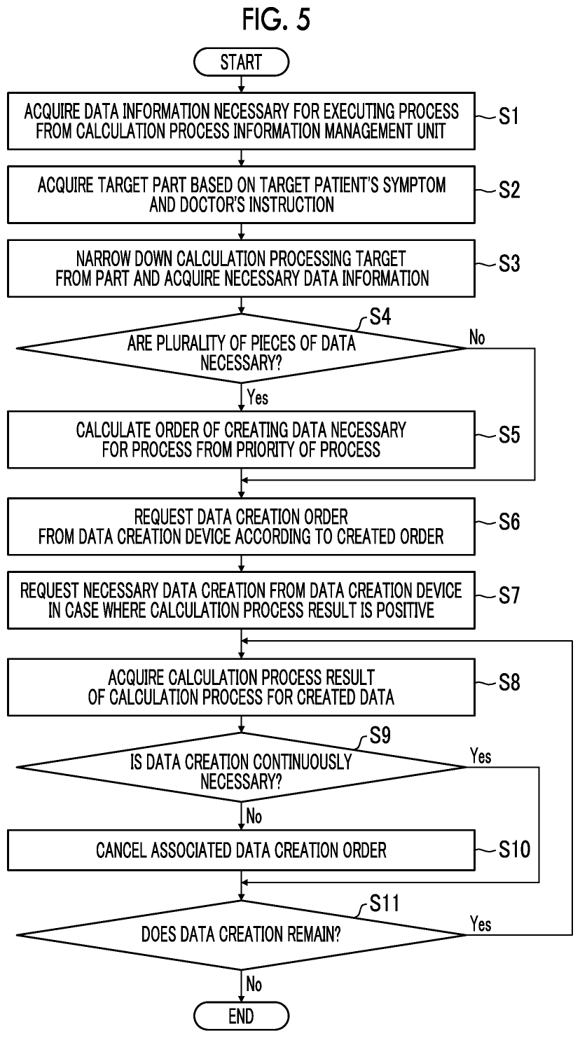

FIG. 5 is a flowchart showing an order management method for medical practices by the order management system 30 for medical practices. The examination reservation management in the hospital is performed by both the order management system 30 for medical practices and the examination reservation system 12. Further, the examination reservation management in the hospital is managed by the operation by the user and the semi-automatic processing.

In Step S1 (an example of a medical practice management step), the medical practice management unit 36 acquires data information necessary for executing the process from a calculation process information management unit. The calculation process information management unit is installed in a calculation processing unit (for example, the AI processing computer 14) that manages resources necessary for AI processing. The calculation processing unit may be disposed on the cloud. Here, the medical practice management unit 36 acquires the table 16 shown in FIG. 2 as data information necessary for executing the process.

In Step S2 (an example of a target part acquisition step), the target part acquisition unit 32 acquires the target part based on the patient's symptom and the doctor's instruction. The target part refers to a part of the human body that performs a medical practice. Here, it is assumed that the “chest” has been acquired as the target part.

In Step S3, the medical practice management unit 36 narrows down the calculation processing target from the target part acquired in Step S2, and acquires the data information necessary for the narrowed down calculation processing target. Here, since the target part is the “chest”, as shown in FIG. 2, the calculation processing target is “bone metastasis AI”, “breast cancer AI”, “lung cancer AI”, and “myocardial infarction AI” using the chest as a target part.

Further, as shown in FIG. 2, in “bone metastasis AI”, the first medical practice is the capturing of a CT image, and the second medical practice is the capturing of a PET image. This “CT image” corresponds to the data information necessary for the calculation process of “bone metastasis AI”.

Similarly, in “breast cancer AI”, “lung cancer AI”, and “myocardial infarction AI”, the first medical practice is CT image capturing, CR image capturing, and electrocardiogram measurement, respectively, and the second medical practice is MG image capturing, CT image capturing, 4 dimensions computed tomography (DCT) image capturing, respectively. Here, the “CT image”, “CR image”, and “electrocardiogram waveform data” correspond to the data information necessary for the calculation process.

In Step S4, the medical practice management unit 36 determines whether or not a plurality of pieces of data are necessary for the narrowed down calculation processing target. In a case where a plurality of data are necessary, the order management system 30 for medical practices performs a process of Step S5. In a case where a plurality of data are not necessary, the order management system 30 for medical practices performs a process of Step S6. Here, since CT images, CR images, and electrocardiogram waveform data are necessary, it is determined that a plurality of pieces of data are necessary.

In Step S5 (an example of an order management step), the medical practice management unit 36 calculates the order of creating data necessary for the calculation process from the priority of the calculation process. The effect and priority of the calculation process are stored in the calculation process information management unit.

In Step S6 (an example of an order management step), the order management unit 38 issues an order for the first medical practice, and requests the issued order from the data creation device 20. In a case where a plurality of pieces of data are necessary for the first medical practice, an order is requested according to the order of creating the data calculated in Step S5. Further, in Step S7 (an example of an order management step), the order management unit 38 issues an order for the second medical practice, and requests the issued order from the data creation device 20. The order management unit 38 may automatically interlock and manage the order for the second medical practice according to a guideline of the disease or the like.

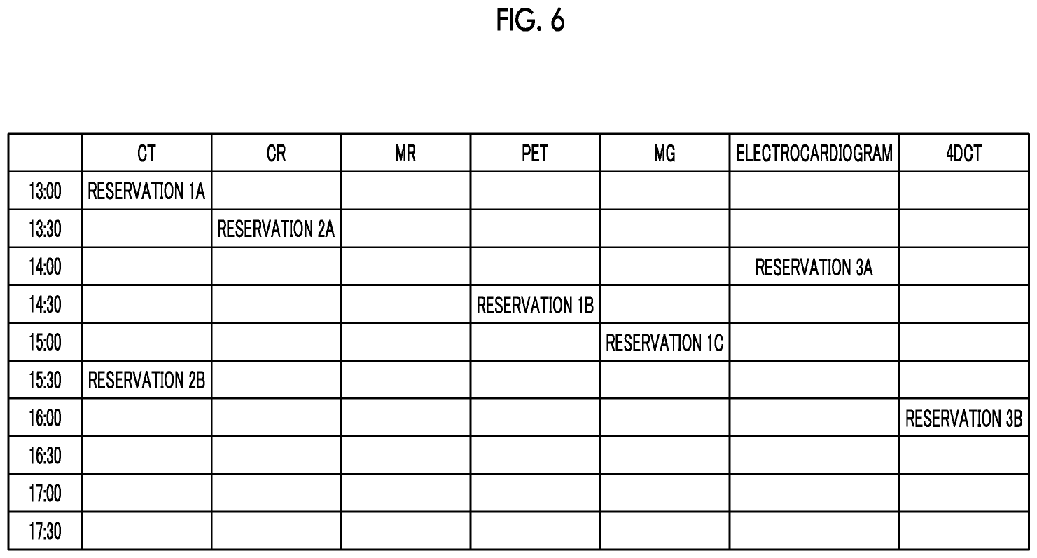

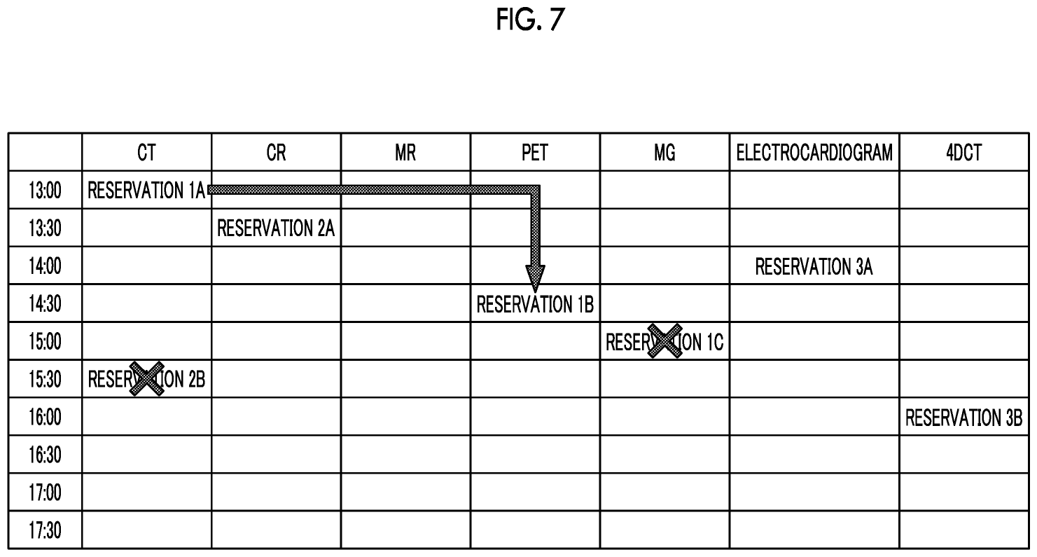

FIGS. 6 and 7 are tables showing orders for each time issued by the order management unit 38 for the patient according to the present embodiment.

As shown in FIG. 6, the order of the CT image is issued as reservation 1A at 13:00. This is an order for the first medical practice of bone metastasis AI and the first medical practice of breast cancer AI.

The order of the CR image is issued as reservation 2A at 13:30. This is an order for the first medical practice of lung cancer AI. Further, the order of the electrocardiogram waveform data is issued as reservation 3A at 14:00. This is an order for the first medical practice of myocardial infarction AI.

In this way, first, data creation orders for a plurality of first medical practices are issued. Accordingly, a plurality of calculation processes can be prioritized over the second medical practice. Here, the order of priority for data creation of the first medical practice is the order of the CT image, the CR image, and the electrocardiogram waveform data. Accordingly, it is possible to give priority to the calculation process having a high priority.

Further, the order of the PET image is issued as reservation 1B at 14:30. This is an order for the second medical practice of bone metastasis AI. Further, the order of the MG image is issued as reservation 1C at 15:00. This is an order for the second medical practice of breast cancer AI.

Furthermore, the order of the CT image is issued as reservation 2B at 15:30. This is an order for the second medical practice of lung cancer AI. Further, the order of the 4DCT image is issued as reservation 3B at 16:00. This is an order for the second medical practice of myocardial infarction AI.

In this way, after the data creation order for the first medical practice, the data creation order for the second medical practice is issued. As will be described later, by giving priority to a plurality of calculation processes over the second medical practice, it is possible to prevent the unnecessary second medical practice from being implemented among the plurality of second medical practices.

The data creation device 20 creates data in order according to the issued order. The AI processing computer 14 performs the corresponding calculation process on the created data by the calculation processing unit 40. For example, for the CT image created by reservation 1A at 13:00, the calculation process is performed by the bone metastasis AI and the breast cancer AI.

In Step S8 (an example of a calculation process result acquisition step), the calculation process result acquisition unit 34 acquires the calculation process result of the calculation process for the data created by the data creation device 20. Here, first, the calculation process results calculated by the bone metastasis AI and the breast cancer AI for the CT image created by reservation 1A at 13:00 are acquired.

In Step S9, the order management unit 38 determines whether or not data creation is continuously necessary based on the calculation process result, that is, the necessity of the second medical practice. In a case where the second medical practice is unnecessary, the order management system 30 for medical practices performs a process of Step S10. In a case where the second medical practice is necessary, the order management system 30 for medical practices performs a process of Step S11.

Here, it is assumed that the calculation process result of bone metastasis AI is positive and the calculation process result of breast cancer AI is negative. In this case, the order management unit 38 determines that the second medical practice is necessary for bone metastasis and that the second medical practice is unnecessary for breast cancer.

In Step S10, the order management unit 38 cancels the associated data creation order. Here, as shown in FIG. 7, the order management unit 38 cancels reservation 1C of the MG image, which is an order for the second medical practice of the breast cancer AI. Therefore, it is possible to prevent the data creation of the MG image, which is an unnecessary second medical practice, from being implemented.

Since the second medical practice is necessary for bone metastasis, the order management unit 38 maintains reservation 1B of the PET image, which is an order for the second medical practice of the bone metastasis AI. In addition, reservation 2B of the CT image, which is an order for the second medical practice of the lung cancer AI, is ordered at 15:30, but reservation 2B may be canceled by using the CT image acquired in reservation 1A at 13:00.

In Step S11, the order management unit 38 determines whether or not the data creation of the first medical practice remains. In a case where the data creation of the first medical practice remains, the calculation process result acquisition unit 34 performs the process of Step S8. In a case where the data creation of the first medical practice does not remain, the order management system 30 for medical practices ends the process of this flowchart.

Here, the calculation process result acquisition unit 34 performs the process of Step S8 again. That is, the calculation process result acquisition unit 34 acquires the calculation process result of the calculation process by the lung cancer AI for the CR image acquired in reservation 2A. Further, in a case where the process of Step S8 is performed again thereafter, the calculation process result acquisition unit 34 acquires the calculation process result of the calculation process by the myocardial infarction AI for the electrocardiogram waveform data acquired in reservation 3A.

As described above, the order management system 30 for medical practices acquires the target part to be examined in the patient, issues an order for the first medical practice and the second medical practice at the acquired target part, acquires the calculation process result of the calculation process regarding the disease executed based on the result of the first medical practice, and determines the necessity of the second medical practice based on the calculation process result. Accordingly, since it is possible to issue an order for a medical practice corresponding to the target part to be examined in the patient, the order for medical practices can be appropriately managed. Here, the order for the second medical practice is canceled in a case where the calculation process result is negative for the disease. Accordingly, it is possible to prevent the implementation of unnecessary medical practices and reduce the amount of data. In addition, the second medical practice may be additionally reserved according to the calculation process result.

### Others

The above order management method for medical practices may be configured as a program for realizing each step on a computer, and a non-transitory recording medium such as a compact disk-read only memory (CD-ROM) that stores this program may also be configured.

In the embodiment described so far, for example, the hardware structure of the processing unit that executes various processes such as the order management system 30 for medical practices is various processors as shown below. The various processors include a central processing unit (CPU) that is a general-purpose processor that functions as various processing units by executing software (program), a programmable logic device (PLD) that is a processor whose circuit configuration can be changed after manufacture, such as a graphics processing unit (GPU) and field programmable gate array (FPGA) which are processors specialized for image processing, a dedicated electrical circuit that is a processor having a circuit configuration designed exclusively for executing a specific process such as an application specific integrated circuit (ASIC), and the like.

One processing unit may be configured by one of various processors, or may be configured by the same or different kinds of two or more processors (for example, a combination of a plurality of FPGAs, a combination of a CPU and an FPGA, or a combination of a CPU and a GPU). In addition, a plurality of processing units may be configured by one processor. As an example of configuring a plurality of processing units by one processor, first, as represented by a computer, such as a server and a client, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more various processors as hardware structures.

Furthermore, the hardware structure of these various processors is, more specifically, an electric circuit (circuitry) in which circuit elements such as semiconductor elements are combined.

The technical scope of the present invention is not limited to the scope described in the above embodiment. The configurations and the like in each embodiment can be appropriately combined between the embodiments without departing from the spirit of the present invention.

### EXPLANATION OF REFERENCES

10: in-hospital system

12: examination reservation system

14: AI processing computer

16: table

18: examination reservation computer

20: data creation device

22: network

30: order management system for medical practices

32: target part acquisition unit

34: calculation process result acquisition unit

36: medical practice management unit

38: order management unit

40: calculation processing unit

60: input layer

62: interlayer

64: output layer

S1 to S11: each step of order management method for medical practice

What is claimed is:

1. An order management method for medical practices, comprising:
a medical practice management step of managing a calculation process regarding a disease, a target part of the calculation process, a first medical practice necessary for executing the calculation process, and a second medical practice necessary for at least one of a diagnosis or treatment of the disease by a doctor in association with each other;
a target part acquisition step of acquiring the target part to be examined in a patient; and
an order management step of issuing an order for the first medical practice and the second medical practice at the acquired target part.

2. The order management method for medical practices according to claim 1, further comprising:
a calculation process result acquisition step of acquiring a calculation process result of the calculation process regarding the disease executed based on a result of the first medical practice,
wherein in the order management step, a necessity of the second medical practice is determined based on the calculation process result.

3. The order management method for medical practices according to claim 2,
wherein in the order management step, the order for the second medical practice is canceled in a case where the calculation process result is negative for the disease.

4. The order management method for medical practices according to claim 1,
wherein in the medical practice management step, the calculation process, the target part, the first medical practice, and the second medical practice are managed in association with each other for each of a plurality of different diseases.

5. The order management method for medical practices according to claim 1,
wherein in the order management step, in a case where there are a plurality of the calculation processes corresponding to the acquired target part, orders for a plurality of first medical practices necessary for executing the plurality of calculation processes are issued in order of priority.

6. The order management method for medical practices according to claim 5,
wherein in the order management step, the order for the second medical practice is issued after the orders for the plurality of first medical practices.

7. The order management method for medical practices according to claim 1,
wherein the calculation process is a process of performing calculation using artificial intelligence.

8. The order management method for medical practices according to claim 7,
wherein the process of performing calculation using the artificial intelligence is a process of outputting an inference result for input data by a learning model trained by machine learning.

9. A non-transitory, tangible computer-readable recording medium which records thereon, a command for causing a computer to execute the order management method according to claim 1, in a case where the command is read by the computer.

10. An order management system for medical practices, comprising:
a medical practice management unit that manages a calculation process regarding a disease, a target part of the calculation process, a first medical practice necessary for executing the calculation process, and a second medical practice necessary for at least one of a diagnosis or treatment of the disease by a doctor in association with each other;
a target part acquisition unit that acquires the target part to be examined in a patient; and
an order management unit that issues an order for the first medical practice and the second medical practice at the acquired target part.

11. A database for medical practices, comprising a calculation process regarding a disease, a target part of the calculation process, a first medical practice necessary for executing the calculation process, and a second medical practice necessary for at least one of a diagnosis or treatment of the disease by a doctor which are stored in association with each other.
